(19) United States
(12) Patent Application Publication
(10) Pub. No.: US 2008/0306410 A1
(43) Pub. Date: Dec. 11, 2008

(54) Methods and apparatuses for measuring pressure points

(75) Inventors: Alex J. Kalpaxis, Glendale, NY (US); David Schieffelin, Woodbury, CT (US); Stacey S. Schieffelin, Woodbury, CT (US); Tracey L. Stetler, Cos Cob, CT (US)

Correspondence Address: VENABLE LLP, P.O. BOX 34385, WASHINGTON, DC, 20043-9998, US

(73) Assignee: 24/8 LLC, Waterbury, CT (US)

(21) Appl. No.: 12/155,558

(22) Filed: Jun. 5, 2008

Related U.S. Application Data
(60) Provisional application No. 60/996,608, filed on Nov. 27, 2007.
(60) Provisional application No. 60/924,931, filed on Jun. 5, 2007.

(51) Int. Cl. A61B 5/103

(52) U.S. Cl. 600/592; 600/587

(57) ABSTRACT

Pressure sensing methods, systems, and computer program products for detecting and monitoring pressure in selectable areas of interest include a sensing system to determine in-sole foot pressure of a user in sports training and monitoring applications.

### CROSS-REFERENCE TO RELATED APPLICATIONS

This application claims priority under 35 U.S.C. § 119(e)(1) of provisional application U.S. Ser. No. 60/924,931, filed Jun. 5, 2007, and provisional application U.S. Ser. No. 60/996,608, filed Nov. 27, 2007.

### BACKGROUND OF THE INVENTION

The following is applicable to pressure sensing methods and systems in general. More particularly, the following relates to detecting insole foot pressure of a user in sports training and monitoring applications, electronic games, and diagnostic systems as will be described with a particular reference thereto. However, it is to be appreciated that the following is also applicable to the other pressure applications.

Athletes utilize various metrics to measure their performance and chart their workouts. The metrics are recorded and analyzed both during and after workouts. For example, interval type workouts typically involve multiple sets of intense activity, semi-intense activity, and rest. The intense activity may be characterized by a range of metrics which correlate to the desired intensity for a particular athlete. Likewise, the rest or semi-intense activity periods may be characterized by a range or metrics which correlate to the desired restful state for a particular athlete.

The human foot combines mechanical complexity and structural strength. The ankle serves as foundation, shock absorber, and propulsion engine. The foot can sustain enormous pressure (i.e., in the range of about several tons over the course of a one-mile run) and provides flexibility and resiliency.

The foot and ankle contain 26 bones (i.e., nearly one-quarter of the bones in the human body are in the feet); 33 joints; more than 100 muscles, tendons (i.e., fibrous tissues that connect muscles to bones), and ligaments (i.e., fibrous tissues that connect bones to other bones); and a network of blood vessels, nerves, skin, and soft tissue.

These components work together to provide the body with support, balance, and mobility. A structural flaw or malfunction in any one part can result in the development of problems elsewhere in the body. Abnormalities in other parts of the body can lead to problems in the feet. Embodiments of the present invention help sense the pressure exerted at a plurality of points of the user's feet to help alleviate such problems.

Structurally, the foot has three main parts: the forefoot, the midfoot, and the hindfoot. The forefoot as shown in FIGS. 2A and 2B is composed of the five toes (called phalanges) and their connecting long bones (metatarsals). Each toe (phalanx) is made up of several small bones. The big toe (also known as the hallux) has two phalanx bones-distal and proximal. It has one joint, called the interphalangeal joint. The big toe articulates with the head of the first metatarsal and is called the first metatarsophalangeal joint (MTPJ for short). Underneath the first metatarsal head are two tiny, round bones called sesamoids. The other four toes each have three bones and two joints. The phalanges are connected to the metatarsals by five metatarsal phalangeal joints at the ball of the foot. The forefoot bears half the body's weight and balances pressure on the ball of the foot.

The midfoot has five irregularly shaped tarsal bones, forms the foot's arch, and serves as a shock absorber. The bones of the midfoot are connected to the forefoot and the hindfoot by muscles and the plantar fascia (arch ligament).

The hindfoot is composed of three joints and links the midfoot to the ankle (talus). The top of the talus is connected to the two long bones of the lower leg (tibia and fibula), forming a hinge that allows the foot to move up and down. The heel bone (calcaneus) is the largest bone in the foot. It joins the talus to form the subtalar joint. The bottom of the heel bone is cushioned by a layer of fat.

A network of muscles, tendons, and ligaments supports the bones and joints in the foot. There are 20 muscles in the foot that give the foot its shape by holding the bones in position and expand and contract to impart movement. The main muscles of the foot are: the anterior tibial, which enables the foot to move upward; the posterior tibial, which supports the arch; the peroneal tibial, which controls movement on the outside of the ankle; the extensors, which help the ankle raise the toes to initiate the act of stepping forward; and the flexors, which help stabilize the toes against the ground. Smaller muscles enable the toes to lift and curl.

There are elastic tissues (tendons) in the foot that connect the muscles to the bones and joints. The largest and strongest tendon of the foot is the Achilles tendon, which extends from the calf muscle to the heel. Its strength and joint function facilitate running, jumping, walking up stairs, and raising the body onto the toes. Ligaments hold the tendons in place and stabilize the joints. The longest of these, the plantar fascia, forms the arch on the sole of the foot from the heel to the toes. By stretching and contracting, it allows the arch to curve or flatten, providing balance and giving the foot strength to initiate the act of walking. Medial ligaments on the inside and lateral ligaments on outside of the foot provide stability and enable the foot to move up and down. Skin, blood vessels, and nerves give the foot its shape and durability, provide cell regeneration and essential muscular nourishment, and control its varied movements.

Pressure sensing methods and systems in particular may be used to detect foot pressure at a plurality of points of the insole of a user engaged in sports training as well as in monitoring applications, electronic games, and diagnostic systems as described in greater detail below.

### BRIEF DESCRIPTION OF THE DRAWINGS

The foregoing and other features of the invention will be apparent from the following, more particular description of exemplary embodiments of the invention, as illustrated in the accompanying drawings wherein like reference numbers generally indicate identical, functionally similar, and/or structurally similar elements. The left most digits in the corresponding reference number indicate the drawing in which an element first appears.

FIG. 1 illustrates a sensing system;

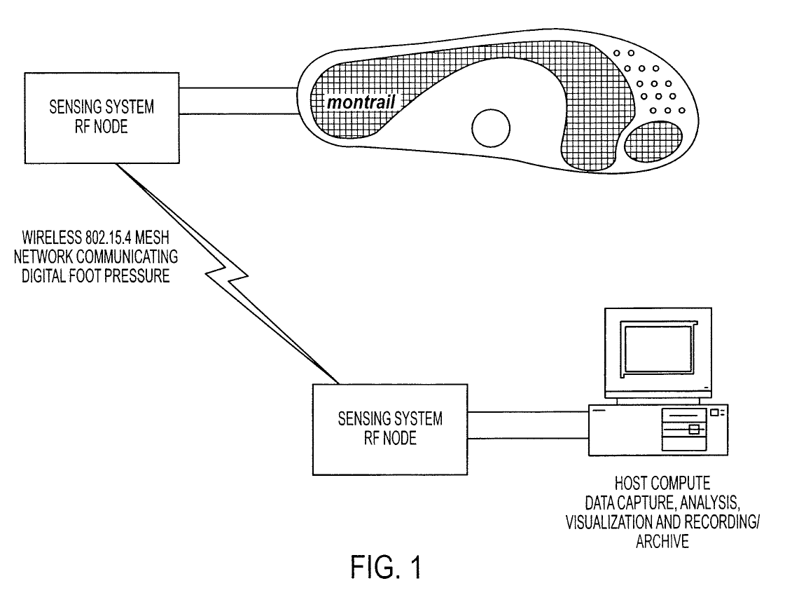

FIGS. 2A and 2B illustrate parts of the human foot;

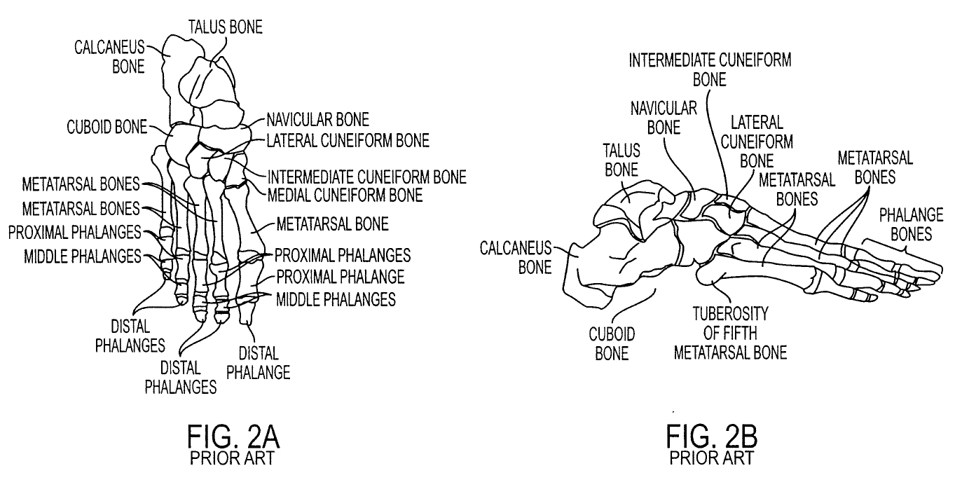

FIG. 3 illustrates a portion of the sensing system;

FIG. 4 illustrates a detailed portion of the transducer;

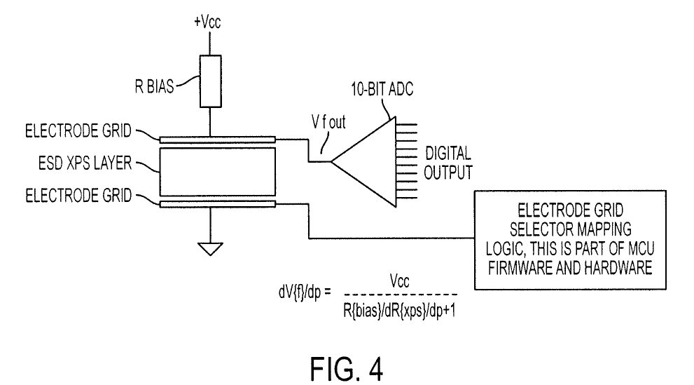

FIG. 5 illustrates data flow from the transducer;

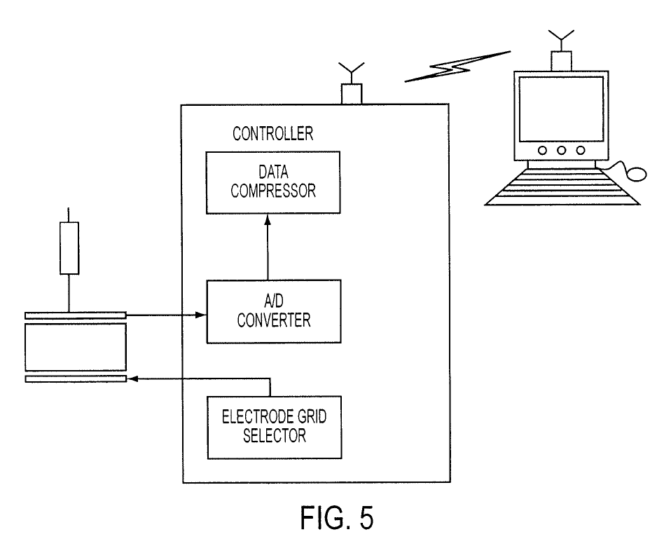

FIG. 6 illustrates an example of a mapping of the transducer;

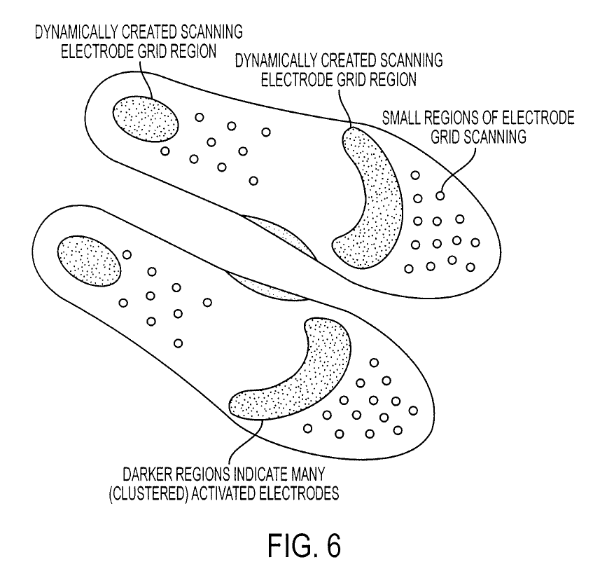

FIG. 7 illustrates an example of a graph showing dependency of the pressure measurement on measured resistance; and

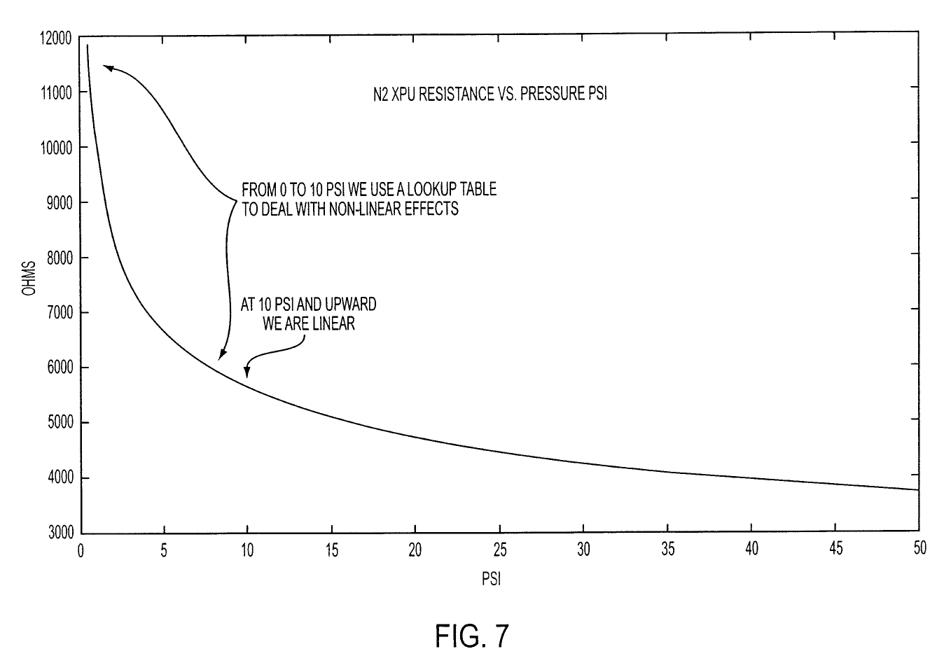

FIG. 8 illustrates a flowchart of the transmission of data.

### DEFINITIONS

In describing the invention, the following definitions may be used throughout (including above).

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and an apparatus that may accept data, may process data in accordance with one or more stored software programs, may generate results, and typically may include input, output, storage, arithmetic, logic, and control units.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet. Exemplary networks may operate with any of a number of protocols, such as Internet protocol (IP), asynchronous transfer mode (ATM), and/or synchronous optical network (SONET), user datagram protocol (UDP), IEEE 802.x, etc.

### DETAILED DESCRIPTION OF EMBODIMENTS OF THE INVENTION

Exemplary embodiments are discussed in detail below. While specific exemplary embodiments are discussed, it should be understood that this is done for illustration purposes only. In describing and illustrating the exemplary embodiments, specific terminology is employed for the sake of clarity. However, the invention is not intended to be limited to the specific terminology so selected. A person skilled in the relevant art may recognize that other components and configurations may be used without parting from the spirit and scope of the invention. It is to be understood that each specific element includes all technical equivalents that operate in a similar manner to accomplish a similar purpose. The examples and embodiments described herein are non-limiting examples.

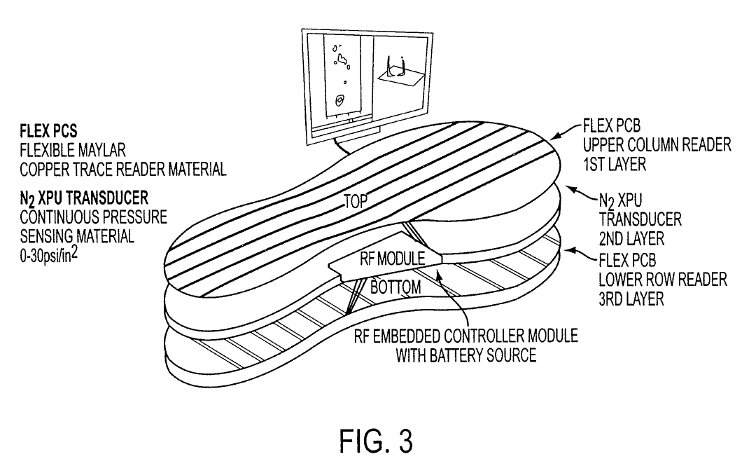

The sensing system insole comprises a foot force transducer that includes a continuous capacitance pressure sensor system. A conventional foot force transducer has a discrete array of capacitors formed by overlapping two sets of conducting strips laid in orthogonal directions on opposite sides of the center layer of a three-layer configuration. See FIG. 3.

The sensing system design allows for flexible placement of conduction elements when creating the typical three-layer configuration. The continuous capacitance pressure sensor elements of the shoe insoles are made using a pressure sensitive variable conductive polymer between conductive traces on sheets of flexible circuit made of a flexible polymer film laminated to a thin sheet of copper that is etched to produce the conductor patterns. This polyimide film is high heat resistance, has dimensional stability, good dielectric strength, with high flexibility, which allows it to survive hostile environments.

The continuous resistive/capacitive sensor layer may be an extruded ESD type ultra high-density conductive XPU foam. This is used to protect against very-high voltage electro-static discharges and provide a compressible form factor for physical device protection against movement shock. The material provides linear resistive and capacitive characteristics through a range of compression forces (0-30 psi). A variable pressure analysis point technique may be used to dynamically map regions of interest for the foot pressure measurement. For instance, in one embodiment, a portion of the heel area and the toe areas may be measured for approximately 10 milliseconds. Next, an arch area may be measured for the 25 milliseconds. This allows for pattern measurements, for instance, in the case of a person with diabetes, where the nerve damage (as a result of the disease) does not allow the person to become aware of the fact that certain areas of the feet are swelling. By using targeted pattern measurement, alerts to changes in plantar foot pressure variations may be provided.

It is contemplated that other materials such as piezoceramic materials which may provide capacitive, piezoelectric, and/or resistive effects may be used.

The sensing system incorporates these modular light-weight, high resolution, continuous pressure sensing shoe sole pads, which are re-configurable for varying arrangements, to wirelessly transmit, detailed pressure data to a host computer, which data is collated and collectively displayed. The sensing system may be integrated with other systems such as vision based sensing systems to provide robust multi-modal sensing capabilities. The sensing system provides a series of applications for data analysis/visualization, data recording and playback. Sensing devices may be grouped together to form clusters that send real-time data to host computers.

The sensing system detects the changes in the electrical properties of continuous capacitance pressure sensors, caused by the mechanical deformation of its material. The sensing system has recording durations of one second at a sampling rate of 50 Hz for a pressure sole that comprises 200 elements results in 10,000 pressure data points per sole per second. With this volume of information, visual presentation and data reduction techniques are used, and the graphical representation of pressure distribution is through wire frame diagrams. These pressure maps are obtained for each sampling interval or at specific instants during the foot-ground contact. A peak pressure graphical representation may be used to illustrate individual foot contact behavior with the ground. This image is created by presenting the highest pressures under the foot, as they have occurred at any time during the ground contact.

The sensing system is able to measure plantar pressure during bipedal standing, which results in about 2.6 times higher heel against forefoot pressures. The highest forefoot pressures are located under the second and third metatarsal heads. There is almost no load sharing contribution of the toes during this standing period. The peak plantar pressures indicate no substantial relationship to body weight. Sensing system measures foot pressures during bipedal standing, walking, and running and shows the highest pressures under the forefoot are found under the third metatarsal head. For bipedal standing as well as walking, peak pressures beneath the third metatarsal head are substantially higher than under the other metatarsal heads. When running, during the impact phase of the ground reaction force, the momentum from the decelerating limb rapidly changes as the foot collides with the ground, resulting in a transient force transmitted up the skeleton. These forces reach magnitudes of up to three times body weight. The repetitive transmission of these forces contributes to degradation and overuse injuries. Sensing system ability to measure plantar pressure distributed over the sole of a foot during running allows for an early determination of potential degradation and overuse injury by profiling the foot's biomechanical characteristics as a result of the impact phase of the ground reaction force.

Sensing system is sensitive enough to measure the plantar pressures differences between adult male and female foot pressures under the longitudinal arch. Under the mid-foot, females have reduced peak foot pressures during standing. Also, for females, there is a correlation between body weight and foot pressures under the longitudinal arch of a female's feet in walking. This allows for the sensing system to analyze the ligamentous structure which results to some degree in collapse of the longitudinal arch during weight bearing phase of walking.

The sensing system is able to perform similar foot function analysis during running. Specifically, the sensing system may analyze midfoot loading as well as the amount of hindfoot rotation which is more apparent in female runners as compared to male runners. In the case for children, contrary to adults, body weight is identified to be of major influence on the magnitude of the pressures under the feet of children and between boys and girls no differences in the foot pressure or relative load patterns are present. The sensing system may be used here periodically to analyze potential walking/running/gait related issues in children as they develop. This may provide data that may help in development of proper in-soles and other support structures to aid in the renormalizing walking/running/gait related issues.

The sensing system may help determine the cause of pain and lower extremity complaints for overweight and obese persons. The system's ability to analyze plantar pressure analysis may provide additional insight into pain and lower extremity complaints. Plantar pressure differences between obese and non-obese adults during standing and walking indicates that the overweight persons have an increase in the forefoot width to foot length ratio. This is due to the broadening of the forefoot under increased weight loading conditions. Even though there is the increased load bearing contact area with the foot against the ground, overweight persons have substantially higher foot pressures under the heel, mid-foot, and forefoot during standing, walking and running.

The sensing system measures larger foot pressures under the midfoot during standing periods for the obese women as compared to the obese men. There is a major influence of body weight on the flattening of the arch is the consequence of the inherent reduced strength of the ligaments in natively in women's feet. This may contribute to lower extremity pain and discomfort in these obese persons and their choice of footwear and predisposition to participation in activities of daily living such as walking and running. For walking, the forefoot pressures as well as the forefoot contact area are substantially increased for obese women. The sensing system may analyze and monitor this increased forefoot plantar pressures, which in most cases result in foot discomfort and hinders these obese women in participating normally in physical activity.

The sensing system may help runners manage overuse injuries; this effects more than half of active runners each year and causes them to stop running. The causes of such injuries include variation/distribution of body dimensions to optimize training, hindfoot movement, kinetic, and strength variables. Biomechanical parameters such as real-time foot pressures are identified and analyzed by the sensing system to help identify key properties of athletic footwear in providing overuse injury protection and performance enhancement. Such parameters may be mid-sole material properties, which may provide information about footwear production tolerances.

The sensing system may measure and record hindfoot rotation, foot pressure patterns, and shock absorption properties running shoes/athletic footwear to analyze shoe characteristics which may help reduce the risk of overuse injuries. The sensing system may be used to evaluate shoe fit and comfort during running on various terrain types. The sensing system's long term monitoring and archive capability allows for analyzing deterioration of shoe properties over time and use.

The sensing system records in real-time in-shoe pressure during running and training and provides information of the interaction between footwear and foot mechanics of the person wearing them. Over rotation during running and training is responsible for many overuse injuries. Typically, restriction of excessive hindfoot motion and improved shock absorption may reduce the risk of running and training injuries. The determination and measurement of subtalar joint rotation are critical the evaluation of running and training shoes. Capturing real-time subtalar joint rotation measurement data is one of the main features of the sensing system.

The sensing system may determine wear and tear with the assessment monitoring and recording features. The sensing system has ability to detect, capture and analyze foot pressure data wirelessly and in real-time variations in hindfoot motion combined with the differences in mid-sole properties to determine shoe cushioning differences to categorize overall stiffness of the shoe. These stiffness characteristic tend to alter the wears landing patterns to elicit lower impact forces. This allows for constructing biomechanical assessments that are beneficial for the wearer using such shoes to minimize injuries resulting from repeated impact loading. The wear of the insole will be displayed outside the shoe as green, yellow, red graphic display indications to illustrate the degree of shoe wear.

The sensing system may perform weight and power assessment by foot zones (heel, mid-foot, and forefoot). The sensing system has capability to detect, capture and analyze foot pressure data wirelessly and in real-time relating to vertical ground reaction force patterns and materials characterization of running shoes with advanced cushioning column systems during walking, running, and/or training.

The sensing system may detect changes in foot sole pressure patterns during activity so that a subject's footfall changes/patterns may be determined during a specific event and correlated against multiple events (practice versus game activity). To be able to detect slight variations of pressure over time—like the loss of fluid within a running race. The ability to transmit this information wirelessly to a collection site or monitor.

The sensing system may detect changes in power patterns during a specific sporting event and calculate power/energy requirements against expected output. Energy vector analysis versus current and expected output.

The sensing system may provide the monitoring and analysis required for dance and kinesiology applications, interactive dance movements—learn to dance as a game application where a subject is signaled in one way when they are taking the right steps and another when they are wrong.

The sensing system may provide the monitoring and analysis required for industrial applications to determine warehouse personnel effectiveness such as allowable personnel movements measured against assembly efficiency, the determination of specific individuals locations (since GPS is not very effective & expensive in-doors, especially in a warehouse setting) to guard against entry into certain areas where they are prohibited such as hazard and/or security areas, and in applications where there are employee health care incentives for weight loss and health maintenance.

The sensing system may augment gaming interfaces to supplement videogames such as PlayStation PS3 and XBox 360 gaming console. This would add an extra dimension to how one interacts with videogames running on these game consoles. Foot pressure activity detected during jumping, walking or running are combined with foot orientation and location data to provide enhance interactivity to the regular popular videogames, allowing for intuitive game play such as kicking or blocking in a fighting game.

The sensing system backend server processing option is able to collect large groups of the sensing system in-sole monitors that would represent a field of players involved in sporting games such as football, soccer, and/or basketball. This may be implemented as a website for remote analysis supporting peer review type applications. The sensing system is able to capture the data over a large field of reference (sports field, field of battle, long distance run) by a specific signature for an individual sole, by person (two soles) or by collection of individuals. To be able to download all of this information upon arrival into transmission zone into a web interface that creates a post event re-simulation to be stored, compared and rated by peer web gamers.

The sensing system backend server processing option is able to collect large groups of the sensing system in-sole monitors that would represent a field of players involved in sporting games such as football, soccer, and/or basketball. This may allow for the creation of game strategy analysis program by using correlation analysis using real-time and archived in-sole data. With additional data input, such as real-time video enhanced dynamic game strategy adjustment programs are possible.

The sensing system is able to detect slight variations of foot pressure over time caused by conditions such as the loss of fluid within a running race, the change in pressure in a medical or rehabilitation environment, the change in pressure during an operating process (driving a car) where pressure may indicate that the operator is fit to continue. With the sensing system monitoring and archive capabilities, programs may be constructed to manage long-term foot pressure variation analysis as previously mentioned.

The system may be implemented in a floor mat type arrangement for a car as the key mechanism for vehicle speed operation. The sensing system may also be used in applications to assist in small motor control where the operator is incapable, either due to injury or birth defect, of applying pressure to hand or foot operating systems. In both cases mentioned, an exemplary embodiment The sensing system wireless support allows for six-degrees of motion.
Features:
Transducer measures resistance & capacitance
Pressure measurements are made by changes in compression of transducer material
Variable column sense and row sense electrode grid capability
Map able row column matrix select pulse generation for data acquisition (analog to digital conversion-ADC)
Fast 32-bit microprocessor enables fast row column electrode scanning at a rate of 25 to 100 complete plantar foot pressure profiles per second.
Product supports both Bluetooth and ZigBee WSN wireless technology
Insole algorithms utilize proprietary efficient compression algorithms for efficient wireless communication.
The product supports in a mesh network configuration up to 65,535 nodes in a 200 meter square area.
Product supports wireless location services with accuracies to 2 meters using unique RSSI algorithms.
The collection node (s) which are attached to host computers collect insole data for real time 3 dimensional viewing.

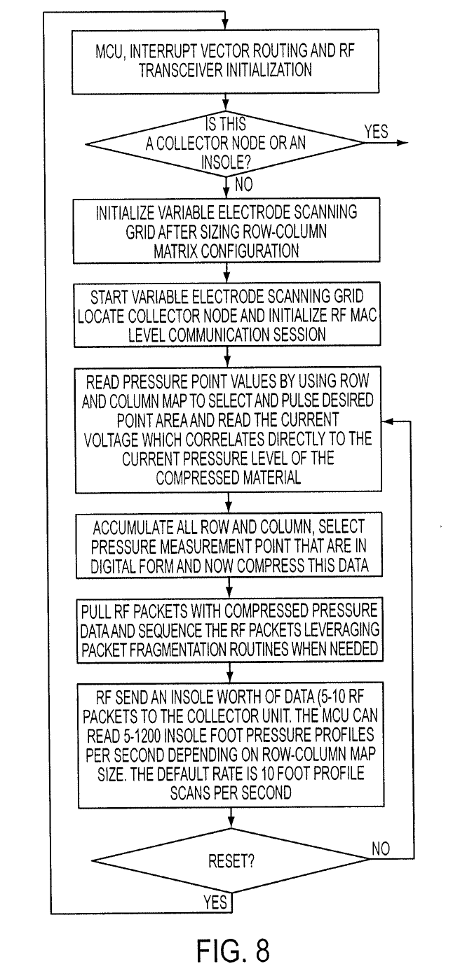

On start up, and referring now to FIG. 8, the sensing system according to embodiments of the present invention will determine if it will be a collector node or an insole node. It does this by determining if any wired interfaces exist, which would be the case if the system was to be a collection node since a USB interface would exist to allow for attachment to a PC.

As a collection node, the sensing system would initialize the MCU, COP, GPIO, SPI, IRQ, and set the desired RF transceiver clock frequency by calling routines MCUInit, GPIOInit, SPIInit, IRQInit, IRQACK, SPIDrvRead, and IRQPinEnable. MCUInit is the master initialization routine which turns off the MCU watchdog, sets the timer module to use BUSCLK as a reference with a pre-scaling of 32. The state variable gu8RTxMode is set to SYSTEM_RESET_MODE and routines GPIOInit, SPIInit and IRQInit are called. Next, the state variable gu8RTxMode is set to RF_TRANSCEIVER_RESET_MODE and the IRQFLAG is check to see if IRQ is asserted. The RF transceiver interrupts are first cleared using SPIDrvRead and then RF transceiver is check for ATTN IRQ interrupts. As a final step for MCUInit, calls are made to PLMEPhyReset (to reset the physical MAC layer), IRQACK (to ACK the pending IRQ interrupt) and IRQPinEnable (to pin Enable, IE, IRQ CLR, on signal's negative edge).

Once the collector node process has been initialized is ready to receive RF packets from insole nodes. This started by creating a RF packet receive queue that is driven by a call back function on RF transceiver packet receive interrupts. When an RF packet is received from an insole node, a check is first made to determine if this from a new insole node or an existing one. If this is from an existing insole node, RF packet sequence numbers are checked to determine continuous synchronization before further analyzing the packet. If this is a new insole node, a insole node context state block is created and initialized. Above this RF packet session level process for node to node communication, is the analysis of the RF packet data payload. This payload contains the compressed plantar foot pressure profile based on the current variable pressure analysis map. The first part of the compressed data contains a map mask array, which is structured as follows:

| 0x10 |00101001|00101101|* * * * |00111101|00101010| 245 | 234 | 219 | 225 | * * * * | 233 |
| start| row 1 | row 2 | | row 15 | row m | D1 | D2 | D3 | D4| |Dn |

Where a bit in the FootMaskArray (row 1, row 2, . . . , row m) is set to one for data that is 255 in value. Each row representation byte uses 6 bits (upper two bits are zero and not used right now) to refer to each A/D channel (there are six in the current utility). Next, the FootRowMask[k] array is scanned for non-active values (no compression). The location in the FootRowMask[k] array where to set the no compression value bit is determined. This is done by first finding out which byte of 16 (which represent rows) in the FootRowMask[k] array is the row that has a no compression value in it. Then remove the base value that brings in the row byte of interest and use the remainder as a bit mask and XOR with existing contents which could be other no compression values already identified.

Once the RF packet from an insole is decompressed the collector node will use the SCITransmitArray routine to send the decompressed RF packet data gsRxPacket.pu8Data and of length gsRxPacket.u8DataLength) to the connected PC host via the USB interface. The insole pressure data is formatted as follow:

|Packet header|0x10| value of A/D CH0|value of A/D CH1|value of A/D CH2|value of A/D CH3|

|value of A/D CH6|value of A/D CH7|value of A/D CH0|value of A/D CH1|
|value of A/D CH2|value of A/D CH3|value of A/D CH6|* * * * *

The IEEE 802.15.4 standard specifies a maximum packet size of 127 bytes and the Time Synchronized Mesh Protocol (TSMP) reserves 47 Bytes for operation, leaving 80 Bytes for payload. The IEEE 802.15.4 is compliant with the 2.4 GHz Industrial, Scientific, and Medical (ISM) band Radio Frequency (RF) transceiver. It contains a complete 802.15.4 Physical layer (PHY) modem designed for the IEEE 802.15.4 wireless standard which supports peer-to-peer, star, and mesh networking. It is combined with a MPU to create the required wireless RF data link and network. The IEEE 802.15.4 transceiver supports 250 kbps O-QPSK data in 5.0 MHz channels and full spread-spectrum encode and decode.

All control, reading of status, writing of data, and reading of data is done through the sensing system node device's RF transceiver interface port. The sensing system node device's MPU accesses the sensing system node device's RF transceiver through interface “transactions” in which multiple bursts of byte-long data are transmitted on the interface bus. Each transaction is three or more bursts long depending on the transaction type. Transactions are always read accesses or write accesses to register addresses. The associated data for any single register access is always 16 bits in length.

Receive mode is the state where the Invention node device's RF transceiver is waiting for an incoming data frame. The packet receive mode allows the Invention node device's RF transceiver to receive the whole packet without intervention from the Invention node device's MPU. The entire packet payload is stored in RX Packet RAM and the micro controller fetches the data after determining the length and validity of the RX packet.

The sensing system node device's RF transceiver waits for preamble followed by a Start of Frame Delimiter. From there, the Frame Length Indicator is used to determine length of the frame and calculate the Cycle Redundancy Check (CRC) sequence. After a frame is received, the Invention device application determines the validity of the packet. Due to noise, it is possible for an invalid packet to be reported with either of the following conditions: A valid CRC and a frame length (0, 1, or 2) and/or Invalid CRC/invalid frame length.

The sensing system node device's application software determines if the packet CRC is valid and that the packet frame length is valid with a value of 3 or greater. In response of the interrupt request from the Invention device RF transceiver, the Invention node device's MPU determines the validity of the frame by reading and checking valid frame length and CRC data. The receive Packet RAM port register is accessed when the Invention node device's RF transceiver is read for data transfer.

The sensing system node device's RF transceiver transmits entire packets without intervention from the Invention node device's MPU. The entire packet payload is pre-loaded in TX Packet RAM, the Invention node device's RF transceiver transmits the frame, and then the transmit complete status is set for the Invention node device's MPU. When the packet is successfully transmitted, transmit interrupt routine that runs on the Invention node device's MPU reports the completion of packet transmission. In response to the interrupt request from the Invention node device's RF transceiver, the Invention node device's MPU reads the status to clear the interrupt and check successful transmission.

Control of the sensing system node device's RF transceiver and data transfers are accomplished by means of a Serial Peripheral Interface (SPI). Although the normal SPI protocol is based on 8-bit transfers, the Invention node device's RF transceiver imposes a higher level transaction protocol that is based on multiple 8-bit transfers per transaction. A singular SPI read or write transaction consists of an 8-bit header transfer followed by two 8-bit data transfers. The header denotes access type and register address. The following bytes are read or write data. The SPI also supports recursive ‘data burst’ transactions in which additional data transfers can occur. The recursive mode is primarily intended for Packet RAM access and fast configuration of the sensing system node device's RF transceiver.

When the invention determines that it is to operate in insole mode, it will reset its state flag, FootStepPacketRecvd and will call its MLMERXEnableRequest routine while enabling a LOW_POWER_WHILE state. The insole node will wait 250 milliseconds for a response from the collector node to determine whether a default full insole electrode scan will be done or a mapped electrode scan will be initiated. In the case of a mapped electrode scan, the collector node send the appropriate electrode scan mapping configuration data. The electrode scanning is performed by the FootScan routine where the FootDataBufferIndex is initialized and rows are activated by enabling MCU direction mode for output [PTCDD_PTCDDN=Output] and bring the associated port line low[PTCD_PTCD6=0]. As each row is activated based on the electrode scanning map, the columns which are attached to the MCU analog signal ports will sample and read the current voltage on the column lines and convert them into digital form which is the plantar foot pressure across that selected row. All rows are sequentially scanned and the entire process repeats until a reset condition or inactivity power-down mode.

The plantar foot pressure data is compressed by clearing the bit map mask array, which is structured as follows:

| 0x10 |00101001|00101101| * * * |00111101|00101010| 245 | 234 | 219 | 225 | * * * | 233 |

|start | row 1| row 2 | * * * | row 15 | row 16 | * * * | row N |Data1|Data2|Data3| * * * |DataN|

This is where a bit in the FootMaskArray[k] is set to one for data that is no compression in value. Each row representation byte uses 6 bits (upper two bits are zero and not used right now) to refer to each A/D channel (there are six). To set the compression bit, call are made to the routine FootSetMask with parameters FootRowMaskIndex and MaskValue set accordingly, which then based on MaskValue an XOR operation is performed on FootRowMask[R] with a selected mask value {0x01; 0x02; 0x04; 0x08; 0x10; 0x20;}.

Several variables such as FootSendNumBytes and FootDataBufferIndex are use to prepare the IEEE 802.15.4 RF packets gsTxPacket.gau8TxDataBuffer[ ] for sending using the compressed data in FootDataBuffer[ ]. The RF packets are sent using the RFSendRequest(&gsTxPacket) routine. This routine checks to see if gu8RTxMode is set at IDLE_MODE and uses gsTxPacket as a pointer to call the RAMDrvWriteTx routine which then calls SPIDrvRead to read the RF transceiver's TX packet length register contents. Using this contents, mask length setting and update and then add 2 for CRC and 2 for code bytes. A call is made to SPIDrvWrite to update the TX packet length field. Next, a call to SPIClearRecieveStatReg is made to clear the status register followed by a call to SPIClearRecieveDataReg to clear the receive data register to make the SPI interface ready for reading or writing.

With the SPI interface ready, a call is made to SPISendChar sending a 0xFF character which represents the 1st code byte. Next, SPIWaitTransferDone is called to verify the send is done.

Now, SPISendChar is called again to send a 0x7E byte, which is the 2nd code byte and then the SPIWaitTransferDone is called again to verify the send is done. With these code bytes sent the rest of the packet is sent using a for loop where psTxPkt->u8DataLength+1 are the number of iterations to a series of sequential to SPISendChar, SPIWaitTransferDone, SPIClearRecieveDataReg. Once this is done, the RF transceiver is loaded with the packet to send. The ANTENNA_SWITCH is set to transmit, the LNA_ON mode enabled and finally a RTXENAssert call made to actually send the packet.

In this manner, by using continuous two dimensional pressure sensing grid with variable mapping capability, the three dimensional real-time planar pressure may be obtained and wirelessly transmitted to a remote location for analysis and display.

While various embodiments of the present invention have been described above, it should be understood that they have been presented by way of example only, and not limitation. For example, the system may be used to sensor fuse with 3-D acceleration data, where correlation will be 3-D motion with foot pressure data. This will allow analysis of caloric expenditure on a real-time basis with virtually 100% accuracy versus now, which is about 90%-95%. Thus, the breadth and scope of the present invention should not be limited by any of the above-described exemplary embodiments, but should instead be defined only in accordance with the following claims and their equivalents.

1. A sensing system, comprising:
a transducer to continually measure pressure of each of a plurality of points in an area of interest, the transducer including:
a compressible layer, and
first and second flexible conductive layers, between which the compressible layer is disposed;
a transmitting/receiving device disposed proximate to the transducer to wirelessly transmit the measured data.

2. The system according to claim 1, wherein each first and second layer includes an electrode grid.

3. The system according to claim 2, further including:
a selector to turn on and off selected points of the electrode grid to variably measure the pressure from the selected points of the area of interest.

4. The system according to claim 1, wherein said plurality of points of interest comprise a plurality of parts of a foot selected from the group consisting of a forefoot area, a midfoot area, and a hindfoot area.

5. The system according to claim 4, wherein said group further comprises one or more of a plurality of phalanges, one or more of a plurality of metatarsals, one or more of a plurality of phalangeal joints, a ball of said foot, one or more of a plurality of tarsal bones forming an arch of said foot, a plantar fascia, a talus, calcaneus, and a subtalar joint.

6. The system according to claim 3, wherein the selector turns on and off the selected points of the electrode grid dynamically in real-time.

7. The system according to claim 1, further including:
a data compressor to compress the measured data before transmitting.

8. The system according to claim 1, wherein the transducer is embedded in a shoe sole.

9. The system according to claim 1, wherein the compressible material comprises a compressible conductive foam.

10. The system according to claim 9, wherein the compressible conductive foam comprises a material suitable for electrostatic discharge (ESD).

11. The system according to claim 1, further including:
a host computer to wirelessly receive the transmitted data and output the received data in a user readable format.

12. The system according to claim 1, further comprising an electronic game coupled to receive the measured data and adapt said game accordingly.

13. The system according to claim 1, further comprising diagnostic means for interpreting the measured data and recommending changes to said pressure points.

14. The system according to claim 13, further comprising an orthotic to make said recommended changes.

15. The system according to claim 1, further comprising tracking means for interpreting the measured data and recommending changes to a training program.
